(19) United States
(12) Patent Application Publication
(10) Pub. No.: US 2026/0078593 A1
(43) Pub. Date: Mar. 19, 2026

(54) Pyramidal Meditation Structure Device

(71) Applicant: Rein Daloul, Anaheim, CA (US)

(72) Inventor: Rein Daloul, Anaheim, CA (US)

(21) Appl. No.: 18/889,094

(22) Filed: Sep. 18, 2024

(51) Int. Cl. E04H 1/12; A61K 41/00; E04B 7/02

(52) U.S. Cl. CPC E04H 1/12; A61K 41/0004; E04B 7/028

(57) ABSTRACT

A pyramidal meditation structure device includes a structure comprised of a plurality of vertical support beams oriented to define a pyramid of sufficient dimensions to house a person. The structure includes a plurality of walls each extending between a respective pair of the vertical support beams. An entrance is integrated into a respective one of the plurality of walls to enable the user to enter or exit the structure. A plurality of pipes is each attached to a respective one of the plurality of vertical support beams such that each of the plurality of pipes is positioned within the structure. A plurality of crystals is each integrated into a respective one of the plurality of pipes to enhance the ornamental appearance of the respective pipe thereby enabling the user to envision each of the sets of crystals as imparting healing energy into the user's meditation.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to meditation structure devices and more particularly pertains to a new meditation structure device for housing a user to practice meditation.

The device includes a structure that has a pyramid shape and an entrance integrated into the structure to enable the user to enter or exit the structure. The device includes a plurality of pipes which are each attached to the structure and positioned within the structure and the plurality of pipes are oriented to define a pyramid. The device includes a plurality of crystals which are each integrated into a respective pipe to enable the user to envision the crystals as imparting healing energy into their meditation.

### (2) Description of Related Art including information disclosed under 37 CFR 1.97 and 1.98

The prior art relates to meditation structure devices including a pyramidal housing device that includes a pyramidal structure comprising a plurality of vertical support beams and plurality of horizontal support beams and a wind generator affixed to the pyramidal structure and a meditation aid device that includes a series of interconnected members that define a pyramidal shape and a penta-tetrahedron shape positioned within the pyramidal shape and an ornamental design for a pyramidal greenhouse that includes a series of interconnected members that are arranged to define a plurality of triangles that are together oriented to define a pyramidal shape. In no instance does the prior art disclose a pyramidal medication structure device that includes a structure comprising a plurality of vertical members and a plurality of walls which together define a pyramidal structure to house a user to practice meditation and a plurality of pipes attached to the vertical members and a plurality of crystals each integrated into respective ones of the plurality of pipes.

### BRIEF SUMMARY OF THE INVENTION

An embodiment of the disclosure meets the needs presented above by generally comprising a structure comprised of a plurality of vertical support beams oriented to define a pyramid of sufficient dimensions to house a person. The structure includes a plurality of walls each extending between a respective pair of the vertical support beams. An entrance is integrated into a respective one of the plurality of walls to enable the user to enter or exit the structure. A plurality of pipes is each attached to a respective one of the plurality of vertical support beams such that each of the plurality of pipes is positioned within the structure. A plurality of crystals is each integrated into a respective one of the plurality of pipes to enhance the ornamental appearance of the respective pipe thereby enabling the user to envision each of the sets of crystals as imparting healing energy into the user's meditation.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective phantom view of a pyramidal meditation structure device according to an embodiment of the disclosure.

FIG. 2 is a cut-away in-use view of an embodiment of the disclosure.

FIG. 3 is a front view of an embodiment of the disclosure.

FIG. 4 is a top phantom view of an embodiment of the disclosure.

FIG. 5 is a cross sectional view taken along line 5-5 of FIG. 1 of an embodiment of the disclosure.

FIG. 6 is a cross sectional view taken along line 6-6 of FIG. 1 of an embodiment of the disclosure.

### DETAILED DESCRIPTION OF THE INVENTION

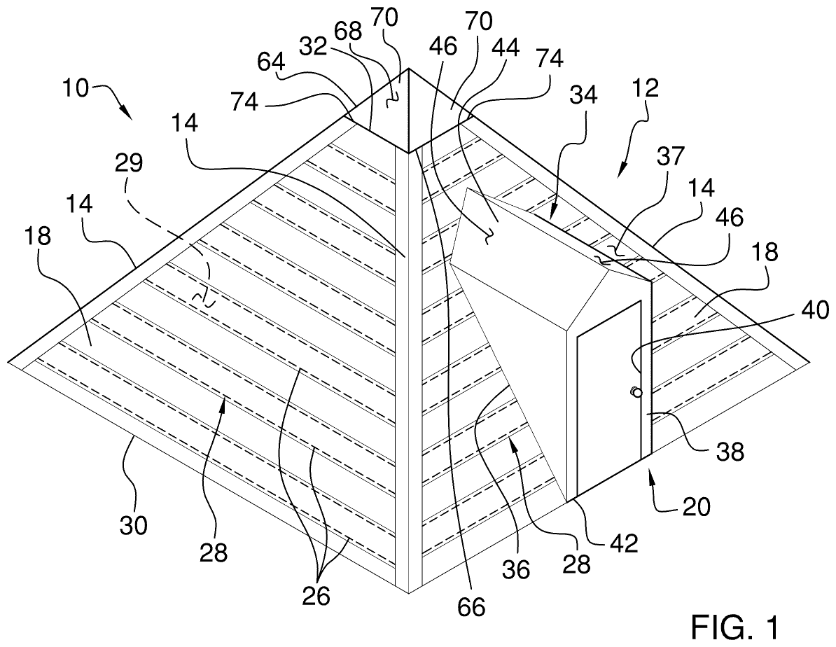

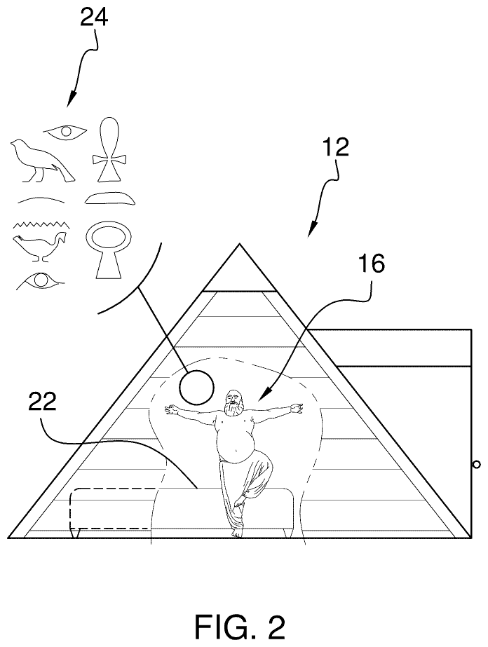

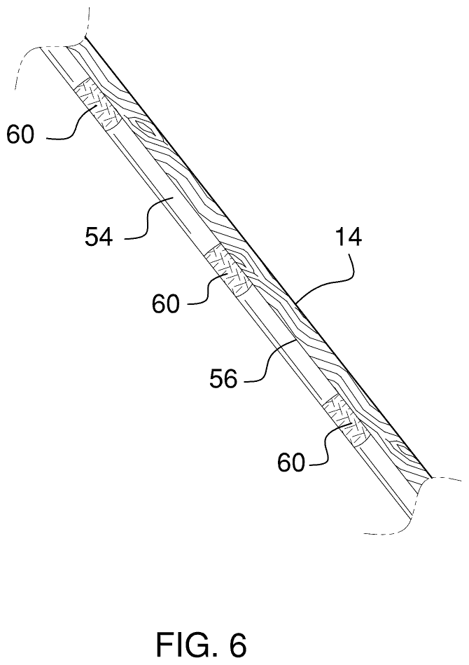

With reference now to the drawings, and in particular to FIGS. 1 through 6 thereof, a new meditation device embodying the principles and concepts of an embodiment of the disclosure and generally designated by the reference numeral 10 will be described.

As best illustrated in FIGS. 1 through 6, the pyramidal meditation structure device 10 generally comprises a structure 12 which is comprised of a plurality of vertical support beams 14 being oriented to define a pyramid having dimensions sufficient to house a person. In this way the structure 12 enables a user 16 to practice meditation within the structure 12. The structure 12 includes a plurality of walls 18 each extending between a respective pair of the vertical support beams 14. Each of the plurality of walls 18 is comprised of a rigid material, including but not being limited to wood or plastic or metal, thereby enabling the plurality of walls 18 to protect the user 16 from weather.

Additionally, the structure 12 has an entrance 20 which is integrated into a respective one of the plurality of walls 18 to enable the user 16 to pass through the entrance 20 to enter or exit the structure 12.

The structure 12 has a height of at least 2.7 meters and the pyramid defined by the structure 12 has an aspect ratio of 1:1.57. In this way the structure 12 has an interior area of at least 21.0 square meters thereby enabling structure 12 to accommodate a queen sized bed 22. In this way the user 16 can sleep within the structure 12 for one or more nights thereby enabling the user 16 to meditate within the structure 12 for extended periods of time. Furthermore, the structure 12 can be employed for glamping, or glamorous camping, and the structure 12 may be outfitted with furniture to enhance the user's 16 comfort for extended periods of time. Indicia 24 are applied to each of the plurality of vertical support beams 14 such that the indicia 24 are visible to the user 16 when the user 16 is inside the structure 12. The indicia 24 on each of the plurality of vertical support beams 14 may comprise images of Egyptian hieroglyphics or other imagery associated with new age meditation and energy healing.

The structure 12 includes a plurality of horizontal support beams 26 and the plurality of horizontal support beams 26 includes a plurality of sets of the horizontal support beams 28. Each of the plurality of sets of horizontal support beams 28 is disposed on an interior surface 29 of a respective one of the plurality of walls 18.

Additionally, each of the plurality of sets of horizontal support beams 28 is attached to and extends between a respective pair of the vertical support beams 14 thereby reinforcing the plurality of vertical support beams 14 in a vertical orientation. The set of horizontal support beams 26 on each of the plurality of walls 18 is evenly spaced apart from each other and is distributed between a bottom edge 30 and a top edge 32 of a respective one of the plurality of walls 18.

The entrance 20 comprises a dormer 34 which has a rear end 36 that is attached to an exterior surface 37 of a respective one of the plurality of walls 18. The dormer 34 has a front end 38 which has an opening 40 extending through the front end 38 to enable the user 16 to pass through the opening 40. A lower edge 42 of the front end 38 is aligned with the bottom edge 30 of the respective wall 18 to which the dormer 34 is attached.

Additionally, the dormer 34 has a roof 44 comprised of a pair of angled surfaces 46 which each extends upwardly from a respective pair of exterior walls 18 of the dormer 34 and intersects at a peak 48.

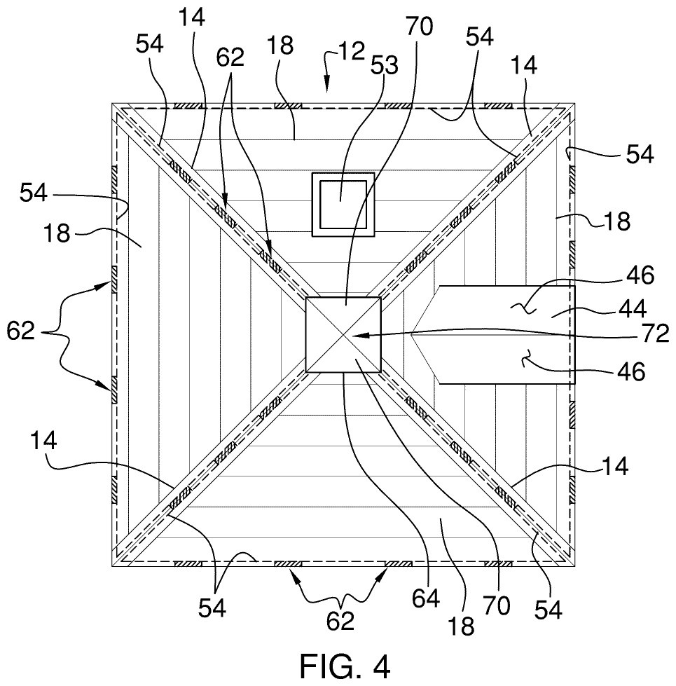

The entrance 20 includes a door 49 which is has a lateral edge 51 that is hingedly coupled to a lateral bounding edge 50 of the opening 40 in the front end 38 of the dormer 34. The door 49 is positionable in a closed position has the door 49 closing the opening 40. Conversely, the door 49 is positionable in an open position having the opening 40 being exposed. The door 49 may include a door knob 52 or other type of mechanism for releasably retaining the door 49 in the closed position. As is shown in FIG. 4, a window 53 is integrated into a respective one of the plurality of walls 18 and the window 53 is comprised of a translucent material, including but not being limited to glass or plastic, to enable light to pass through the window 53 and illuminate the interior of the structure 12. Additionally, the window 53 and the entrance 20 are positioned on unique walls 18 with respect to each other.

A plurality of pipes 54 is included and each of the plurality of pipes 54 is attached to an outer surface 56 of a respective one of the plurality of vertical support beams 14 such that each of the plurality of pipes 54 is positioned within the structure 12.

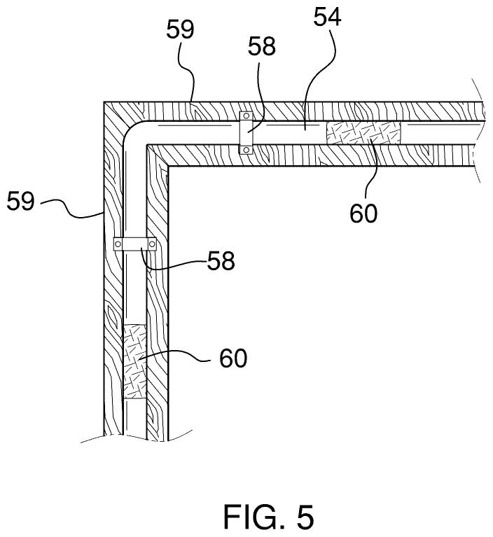

Furthermore, each of the plurality of pipes 54 extends along a full length of the respective vertical support beam 14. In this way the plurality of pipes 54 defines a pyramid shape within the structure 12. As is most clearly shown in FIG. 5, a series of pipe clamps 58, or other types of fasteners, may be provided which serve to mount each of the plurality of pipes 54 to the respective vertical support beam 14. As is shown in FIG. 5, respective ones of the plurality of pipes 54 is each attached to a bottommost one 59 of each of the sets of horizontal support beams 28 to define a base of the pyramid shape defined by the plurality of pipes 54.

A plurality of crystals 60 is included and the plurality of crystals 60 includes a plurality of sets of the crystals 62. Each of the plurality of sets of crystals 62 is integrated into a respective one of the plurality of pipes 54 to enhance the ornamental appearance of the respective pipe 54. Furthermore, each of the plurality of crystals 60 may be comprised of Quartz, Amethyst, Tourmaline or other types of crystals commonly associated with new age meditation and energy healing. In this way the plurality of crystals 60 enable the user 16 to envision each of the sets of crystals 62 as imparting healing energy into the user's 16 meditation. Each of the plurality of sets of crystals 62 is evenly spaced apart from each other and is distributed along a full length of the respective pipe 54. Furthermore, each of the plurality of sets of crystals 62 is structured to define a cylindrical shape thereby enabling each of the plurality of sets of crystals 62 to conform to the shape of the respective pipe 54.

A roof cap 64 is provided which has a basal surface 66 and an exterior surface 68 extending upwardly from the basal surface 66; the exterior surface 68 of the roof cap 64 has a plurality of intersecting sides 70 each angling inwardly from the basal surface 66. Each of the plurality of intersecting sides 70 of the exterior surface 68 of the roof cap 64 terminates at a peak 72 which is spaced from the basal surface 66 such that the roof cap 64 has a pyramidal shape. The basal surface 66 is attached to an upper end 74 of each of the plurality of vertical support beams 14 such that each of the plurality of intersecting sides 70 of the exterior surface 68 of the roof cap 64 lies on a plane which is oriented coplanar with the exterior surface 37 of a respective one of the plurality of walls 18 of the structure 12. Furthermore, the roof cap 64 is comprised of a rigid material, including but not being limited to copper or other metal commonly associated with healing energy for new age meditation.

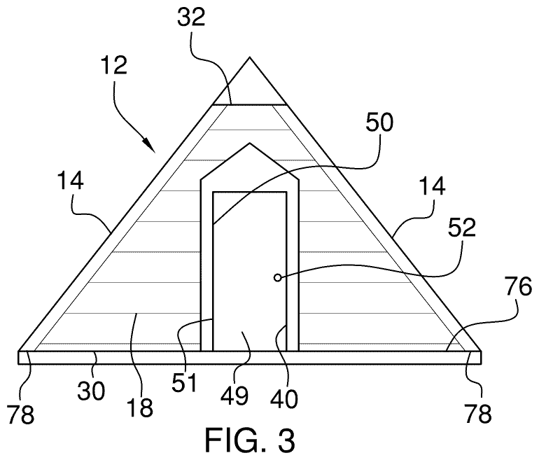

As is shown in FIG. 3, the structure 12 includes a foundation 76, which might be comprised of poured concrete or wood framing, upon which the structure 12 is positioned. The bottommost one 59 of each of the sets of horizontal support beams 28 is attached to the foundation 76 with fasteners, including but not being limited to penetrating bolts or bracket, to ensure that the structure 12 is secured to the foundation 76 to resist shifting on the foundation 76. A lower end 78 of each of the vertical support beams 14 may also be fastened to the foundation 76 to enhance the structural integrity of the structure 12. Each of the plurality of walls 18 may be stepped such that the structure 12 has the ornamental appearance of a Mayan pyramid.

In use, the structure 12 is positioned outdoors to enable the user 16 to occupy the structure 12 for extended periods of time in the convention of glamping, otherwise known as glamorous camping. The dimensions of the structure 12 enable the user 16 to practice meditation within the structure 12 along with accommodating a queen sized bed 22 and other furniture. In this way the user 16 can enjoy the comforts of home while the user 16 is occupying the structure 12. The pyramidal shape of the structure 12 as well as the pyramidal shape defined by the plurality of pipes 54 enables the user 16 to envision the healing energy that is commonly associated with pyramids in new age meditation. Furthermore, the plurality of crystals 60 enables the user 16 to envision the healing energy commonly associated with crystals in new age meditation. In this way the structure 12 and the crystals 60 enable the user 16 to enjoy all of the benefits of new age meditation with respect to healing energy and an overall sense of well-being.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A pyramidal meditation structure device for housing an individual practicing meditation, said device comprising:
a structure comprised of a plurality of vertical support beams being oriented to define a pyramid having dimensions sufficient to house a person wherein said structure is configured to enable a user to practice meditation within said structure, said structure having a height of at least 2.7 meters, said pyramid defined by said structure having an aspect ratio of 1:1.57 thereby enabling said structure to have an interior area of at least 21.0 square meters wherein said structure is configured to accommodate a queen sized bed thereby enabling the user to sleep within said structure, said structure including a plurality of walls each extending between a respective pair of said vertical support beams, said structure having an entrance being integrated into a respective one of said plurality of walls wherein said entrance is configured to enable the user to pass through said entrance to enter or exit said structure;
a plurality of pipes, each of said plurality of pipes being attached to a respective one of said plurality of vertical support beams such that each of said plurality of pipes is positioned within said structure, each of said plurality of pipes extending along a full length of said respective vertical support beam; and
a plurality of crystals, said plurality of crystals including a plurality of sets of said crystals, each of said plurality of sets of crystals being integrated into a respective one of said plurality of pipes wherein each of said plurality of sets of crystals is configured to enhance the ornamental appearance of said respective pipe thereby enabling the user to envision each of said sets of crystals as imparting healing energy into the user's meditation.

2. The device according to claim 1, wherein:
said structure includes a plurality of horizontal support beams;
said plurality of horizontal support beams includes a plurality of sets of said horizontal support beams;
each of said plurality of sets of horizontal support beams is disposed on an interior surface of a respective one of said plurality of walls;
each of said plurality of sets of horizontal support beams is attached to and extends between a respective pair of said vertical support beams thereby reinforcing said plurality of vertical support beams in a vertical orientation; and
said set of horizontal support beams on each of said plurality of walls is evenly spaced apart from each other and is distributed between a bottom edge and a top edge of a respective one of said plurality of walls.

3. The device according to claim 1, wherein:
said entrance includes a dormer having a rear end being attached to an exterior surface of a respective one of said plurality of walls;
said dormer has a front end which has an opening extending through said front end wherein said opening is configured to enable the user to pass through said opening;
a lower edge of said front end is aligned with said bottom edge of said respective wall to which said dormer is attached;
said dormer has a roof comprised of a pair of angled surfaces which each extends upwardly from a respective pair of exterior walls of said dormer and intersect at a peak; and
said entrance includes a door being having a lateral edge being hingedly coupled to a lateral bounding edge of said opening in said front end of said dormer;
said door is positionable in a closed position having said door closing said opening; and
said door is positionable in an open position having said opening being exposed.

4. The device according to claim 1, wherein:
each of said plurality of sets of crystals is evenly spaced apart from each other and is distributed along a full length of said respective pipe; and
each of said plurality of sets of crystals is structured to define a cylindrical shape thereby enabling each of said plurality of sets of crystals to conform to the shape of said respective pipe.

5. The device according to claim 1, wherein:
said device includes a roof cap having a basal surface and an exterior surface extending upwardly from said basal surface;
said exterior surface has a plurality of intersecting sides each angling inwardly from said basal surface; and
each of said plurality of intersecting sides of said exterior surface terminates at a peak being spaced from said basal surface such that said roof cap has a pyramidal shape.

6. The device according to claim 5, wherein said basal surface is attached to an upper end of each of said plurality of vertical support beams such that each of said plurality of intersecting sides of said exterior surface lies on a plane being oriented coplanar with said exterior surface of a respective one of said plurality of walls of said structure.

7. A pyramidal meditation structure device for housing an individual practicing meditation, said device comprising:
a structure comprised of a plurality of vertical support beams being oriented to define a pyramid having dimensions sufficient to house a person wherein said structure is configured to enable a user to practice meditation within said structure, said structure having a height of at least 2.7 meters, said pyramid defined by said structure having an aspect ratio of 1:1.57 thereby enabling said structure to have an interior area of at least 21.0 square meters wherein said structure is configured to accommodate a queen sized bed thereby enabling the user to sleep within said structure, said structure including a plurality of walls each extending between a respective pair of said vertical support beams, said structure having an entrance being integrated into a respective one of said plurality of walls wherein said entrance is configured to enable the user to pass through said entrance to enter or exit said structure, said structure including a plurality of horizontal support beams, said plurality of horizontal support beams including a plurality of sets of said horizontal support beams, each of said plurality of sets of horizontal support beams being disposed on an interior surface of a respective one of said plurality of walls, each of said plurality of sets of horizontal support beams being attached to and extending between a respective pair of said vertical support beams thereby reinforcing said plurality of vertical support beams in a vertical orientation, said set of horizontal support beams on each of said plurality of walls being evenly spaced apart from each other and being distributed between a bottom edge and a top edge of a respective one of said plurality of walls, said entrance comprising:
a dormer having a rear end being attached to an exterior surface of a respective one of said plurality of walls, said dormer having a front end which has an opening extending through said front end wherein said opening is configured to enable the user to pass through said opening, a lower edge of said front end being aligned with said bottom edge of said respective wall to which said dormer is attached, said dormer having a roof comprised of a pair of angled surfaces which each extends upwardly from a respective pair of exterior walls of said dormer and intersect at a peak; and
a door being having a lateral edge being hingedly coupled to a lateral bounding edge of said opening in said front end of said dormer, said door being positionable in a closed position having said door closing said opening, said door being positionable in an open position having said opening being exposed;
a plurality of pipes, each of said plurality of pipes being attached to an outer surface of a respective one of said plurality of vertical support beams such that each of said plurality of pipes is positioned within said structure, each of said plurality of pipes extending along a full length of said respective vertical support beam;
a plurality of crystals, said plurality of crystals including a plurality of sets of said crystals, each of said plurality of sets of crystals being integrated into a respective one of said plurality of pipes wherein each of said plurality of sets of crystals is configured to enhance the ornamental appearance of said respective pipe thereby enabling the user to envision each of said sets of crystals as imparting healing energy into the user's meditation, each of said plurality of sets of crystals being evenly spaced apart from each other and being distributed along a full length of said respective pipe, each of said plurality of sets of crystals being structured to define a cylindrical shape thereby enabling each of said plurality of sets of crystals to conform to the shape of said respective pipe; and
a roof cap having a basal surface and an exterior surface extending upwardly from said basal surface, said exterior surface of said roof cap having a plurality of intersecting sides each angling inwardly from said basal surface, each of said plurality of intersecting sides of said exterior surface of said roof cap terminating at a peak being spaced from said basal surface such that said roof cap has a pyramidal shape, said basal surface being attached to an upper end of each of said plurality of vertical support beams such that each of said plurality of intersecting sides of said exterior surface of said roof cap lies on a plane being oriented coplanar with said exterior surface of a respective one of said plurality of walls of said structure.
